(19) United States
(12) Patent Application Publication
(10) Pub. No.: US 2007/0106534 A1
(43) Pub. Date: May 10, 2007

(54) COMPUTERIZED SYSTEM AND METHOD FOR PREDICTING AND TRACKING BILLING GROUPS FOR PATIENTS IN A HEALTHCARE ENVIRONMENT

(75) Inventors: ANNE MOLINARO, KANSAS CITY, MO (US); HEATHER M. MILLER, KANSAS CITY, MO (US)

Correspondence Address: SHOOK, HARDY & BACON L.L.P.;Intellectual Property Department, 2555 GRAND BOULEVARD, KANSAS CITY, MO, 64108-2613, US

(73) Assignee: CERNER INNOVATION, INC., OVERLAND PARK, KS (US)

(21) Appl. No.: 11/558,225

(22) Filed: Nov. 9, 2006

Related U.S. Application Data
(60) Provisional application No. 60/735,031, filed on Nov. 9, 2005.

(51) Int. Cl. G06Q 10/00; A61B 5/00

(52) U.S. Cl. 705/2; 600/300

(57) ABSTRACT

A computerized system and method in a healthcare environment for calculating one or more predicted billing groups for a patient is provided. One or more data elements for a patient are received prior to the patient being discharged from a healthcare facility. The one or more data elements are utilized to calculate one or more predicted billing groups for the patient. The one or more predicted billing groups for the patient are stored.

### CROSS-REFERENCE TO RELATED APPLICATIONS

This application claims the benefit of priority to U.S. Provisional Application Ser. No. 60/735,031, filed on Nov. 9, 2005.

### STATEMENT REGARDING FEDERALLY SPONSORED RESEARCH OR DEVELOPMENT

Not applicable.

### BACKGROUND

As healthcare costs began to escalate, in 1983, the retrospective payment system for the Medicare program was replaced a prospective payment system. The prospective payment system pays for acute hospital care based on the expected costs, rather than accrued charges. Each patient discharged from a hospital setting is categorized into a billing group called a Diagnosis Related Group (DRG). The DRGs are the patient classification system that facilitates prospective payment to hospitals.

The International Classification of Diseases, Ninth Revision, Clinical Modifications (ICD-9-CM) is used to implement the DRG prospective payment system. ICD-9-CM is a diagnostic dictionary allowing diseases, symptoms, health problems and procedures to be classified and coded. The coded data elements are utilized to determine the DRG for a patient after the patient is discharged. Generally, the hospital is then paid a flat fee for the patient's stay based on the patient's calculated DRG regardless of the services and actual resources provided. Generally the flat fee payment represents the average cost for caring for a patient within a particular DRG. Along with Medicare, some private insurance companies use DRGs to calculate the amount of reimbursement for a patient's stay in a healthcare facility.

Billing groups for financial reimbursement may be used for both inpatient and outpatient stays in a healthcare facility. Other billing groups used in the United States include ambulatory payment classification codes (APC) used for outpatient treatment, such as one-day surgeries. Internationally, a variety of billing groups may also be used, including German billing groups (DDRG) and United Kingdom billing groups (HRG). Currently, however, billing groups are calculated at or after discharge of a patient from a healthcare facility.

It would beneficial to have a system and method to calculate and track predicted billing groups for one or more patients from the time of the admission and during treatment at a healthcare facility.

### SUMMARY

In one embodiment of the present invention, a method in a computerized healthcare environment for calculating one or more predicted billing groups for a patient is provided. One or more data elements for a patient are received prior to the patient being discharged from a healthcare facility. The one or more data elements are utilized to calculate one or more predicted billing groups for the patient. The one or more predicted billing groups for the patient are stored.

In another embodiment, a method in a computerized healthcare environment for calculating one or more final billing groups for a patient is provided. One or more predicted billing groups for a patient are accessed and are utilized for calculating one or more final billing groups for the patient.

In still another embodiment, a computer system healthcare environment for calculating one or more predicted billing groups for a patient is provided. The computer system comprises a receiving component for receiving one or more data elements for a patient prior to the patient being discharged from a healthcare facility and a utilizing component for utilizing the one or more data elements to calculate one or more predicted billing groups for the patient. The computer system further comprises a storing component for storing the one or more calculated billing groups for the patient.

In yet another embodiment, a computer system in a healthcare environment for calculating one or more final billing groups for a patient is provided. The computer system comprises an accessing component for accessing one or more predicted billing groups for a patient and a utilizing component for utilizing the one or more predicted billing groups for the patient for calculating one or more final billing groups for the patient.

### BRIEF DESCRIPTION OF THE SEVERAL VIEWS OF THE DRAWINGS

The present invention is described in detail below with reference to the attached drawing figures, wherein:

FIG. 1 is a block diagram illustrating a system for use in accordance with an embodiment of the present invention;

FIG. 2 is a block diagram illustrating a database for use in accordance with an embodiment of the present invention;

FIG. 3 is a flow diagram illustrating a method for calculating and storing predicted billing groups and related data in accordance with an embodiment of the present invention;

FIG. 4 is flow diagram illustrating a method for recalculating predicted billing groups in accordance with an embodiment of the present invention;

FIG. 5 is a flow diagram illustrating a method for calculating a final billing group utilizing the predicted billing group in accordance with an embodiment of the present invention; and

FIG. 6 is a screen displaying an order documentation form displaying a predicted billing group in accordance with an embodiment of the present invention.

### DETAILED DESCRIPTION

In one embodiment of the present invention, billing groups utilized for financial reimbursement are calculated at the time of admission to drive reimbursement upon discharge of a patient. A window into the financial side of healthcare treatment is provided throughout the patient's care in the healthcare facility. An integrated workflow between a clinical system and financial system is provided. Furthermore, a history for the calculation and progression of a predicted billing group throughout a patient's stay is provided. A predicted billing group for a patient may be calculated or recalculated at any point during the patient's healthcare stay before the patient is discharged. Furthermore, the calculation of a predicted billing group at the time of the admission may also set forth a clinical pathway for the patient and drive the healthcare of the patient during their stay.

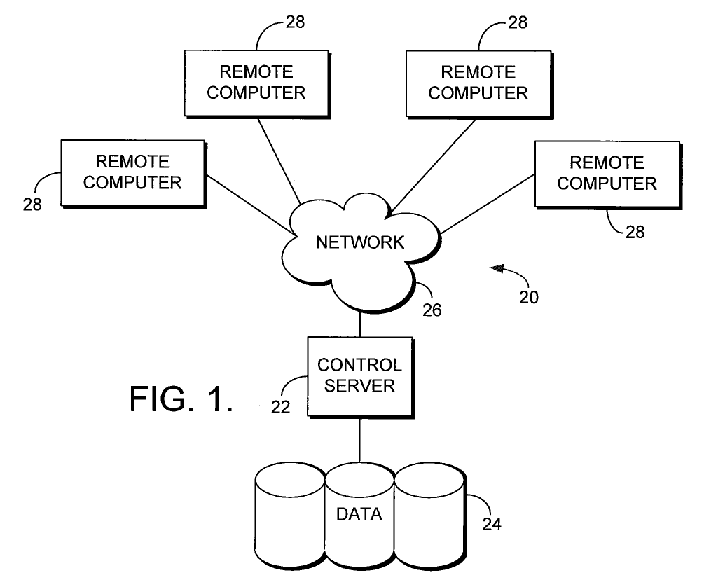

With reference to FIG. 1, an exemplary medical information system for implementing embodiments of the invention includes a general purpose-computing device in the form of server 22. Components of server 22 may include, but are not limited to, a processing unit, internal system memory, and a suitable system bus for coupling various system components, including database cluster 24 to the control server 22. The system bus may be any of several types of bus structures, including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Server 22 typically includes therein or has access to a variety of computer readable media, for instance, database cluster 24. Computer readable media can be any available media that can be accessed by server 22, and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information, such as computer readable instructions, data structures, program modules or other data. Computer storage media includes, but is not limited to, RAM, ROM, EEPROM, flash memory or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by server 22. Communication media typically embodies computer readable instructions, data structures, program modules, or other data in a modulated data signal, such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media, such as a wired network or direct-wired connection, and wireless media such as acoustic, RF, infrared and other wireless media. Combinations of any of the above should also be included within the scope of computer readable media.

The computer storage media, including database cluster 24, discussed above and illustrated in FIG. 1, provide storage of computer readable instructions, data structures, program modules, and other data for server 22.

Server 22 may operate in a computer network 26 using logical connections to one or more remote computers 28. Remote computers 28 can be located at a variety of locations in a medical or research environment, for example, but not limited to, clinical laboratories, hospitals, other inpatient settings, a clinician's office, ambulatory settings, medical billing and financial offices, hospital administration, veterinary environment and home health care environment. Clinicians include, but are not limited to, the treating physician, specialists such as surgeons, radiologists and cardiologists, emergency medical technologists, physician's assistants, nurse practitioners, nurses, nurse's aides, pharmacists, dieticians, microbiologists, laboratory experts, laboratory scientist, laboratory technologists, genetic counselors, researchers, veterinarians and the like. The remote computers may also be physically located in non-traditional medical care environments so that the entire health care community is capable of integration on the network. Remote computers 28 may be a personal computer, server, router, a network PC, a peer device, other common network node or the like, and may include some or all of the elements described above relative to server 22. Computer network 26 may be a local area network (LAN) and/or a wide area network (WAN), but may also include other networks. Such networking environments are commonplace in offices, enterprise-wide computer networks, intranets and the Internet. When utilized in a WAN networking environment, server 22 may include a modem or other means for establishing communications over the WAN, such as the Internet. In a networked environment, program modules or portions thereof may be stored in server 22, or database cluster 24, or on any of the remote computers 28. For example, and not limitation, various application programs may reside on the memory associated with any one or all of remote computers 28. It will be appreciated that the network connections shown are exemplary and other means of establishing a communications link between the computers may be used.

A user may enter commands and information into server 22 or convey the commands and information to the server 22 via remote computers 28 through input devices, such as keyboards, pointing devices, commonly referred to as a mouse, trackball, or touch pad. Other input devices may include a microphone, scanner, or the like. Server 22 and/or remote computers 28 may have any sort of display device, for instance, a monitor. In addition to a monitor, server 22 and/or computers 28 may also include other peripheral output devices, such as speakers and printers.

Although many other internal components of server 22 and computers 28 are not shown, those of ordinary skill in the art will appreciate that such components and their interconnection are well known. Accordingly, additional details concerning the internal construction of server 22 and computer 28 need not be disclosed in connection with the present invention. Although the method and system are described as being implemented in a LAN operating system, one skilled in the art would recognize that the method and system can be implemented in any system.

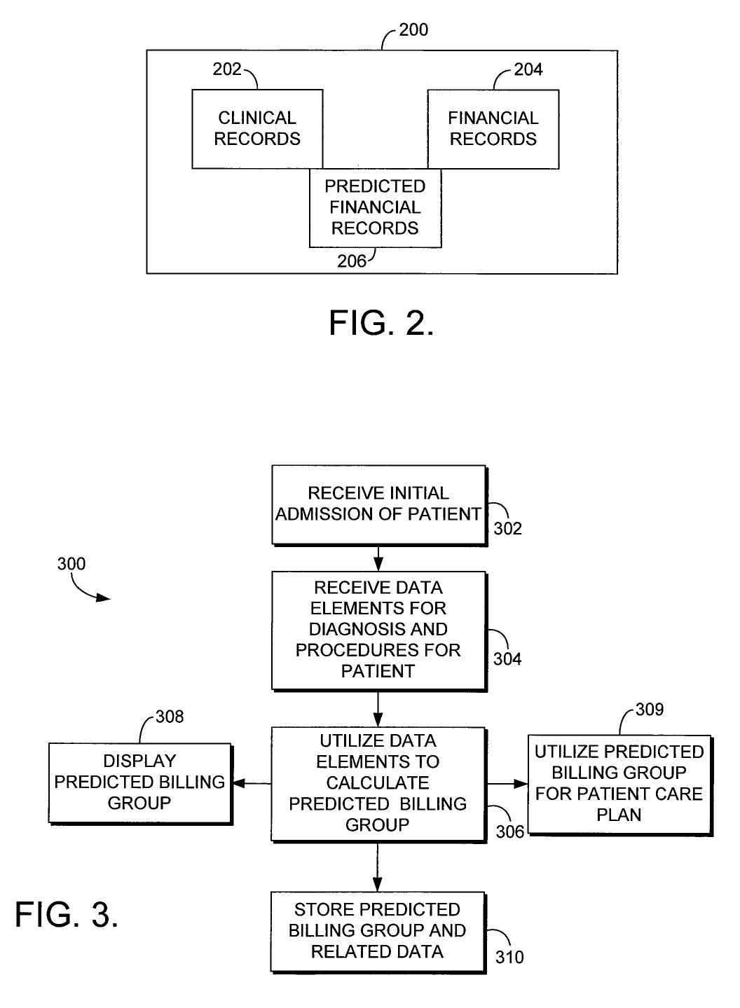

With reference to FIG. 2, a computerized database 200 that may be used with an embodiment of the present invention is shown. The database contains clinical records 202 for a patient, financial records 204 for a patient, and predicted financial records 206 for a patient. Clinical records 202 may include treatment history for a patient, patient diagnosis, demographic information including age and sex, orders entered by a physician for treatment of a patient, and a variety of clinical information related to the patient including estimated and actual length of stay for the patient, planned and completed procedures for the patient and the disposition of patient at discharge. Financial records 204 may include financial information for the patient including final billing groups, invoices, payment history, insurance information and other financial information related to a patient's account. Predicted financial records include predicted billing groups for patients and historic information related to the calculation of predicted billing groups for patients. One of skill in the art will appreciate that clinical records 202, financial records 204 and predicted financial records for a patient may be contained in one computer database such as database 200 or may be contained in multiple databases.

With reference to FIG. 3, a method is shown for calculating and storing a predicted billing group. A predicted billing group may include such groups as diagnosis related groups (DRG), German billing groups (DDRG), United Kingdom billing groups (HRG), and ambulatory payment classification codes (APC). At step 302, data indicating the initial admission of a healthcare patient are received. Upon the admission and initial assessment of a patient, data elements for the patient will be entered by healthcare providers and are received by the system.

At step 304, the data elements to be utilized to calculate a predicted billing group for the patient are received. Exemplary data elements that may be utilized for calculating a predicted billing group include the estimated length of stay for the patient, admitting primary and secondary diagnosis codes, details associated with planned and performed procedures, surgeries and tests, and the age and gender of patient. At step 306, the data elements are utilized to calculate one or more predicted billing groups for the patient. In other words, a billing group is determined using the currently available data in the system by one of many algorithms or grouping calculators well known by one of skill in the art. In one example, the predicted group is determined by calculating the group using existing data elements in the system rather than the full complement of data elements that will subsequently become available prior to discharge. In another example, the clinician may predict particular data elements such as length of stay, and a predicted grouper may be determined based on this prediction and the known data elements. In another example, a predicted length of stay may be determined based on predictive models and algorithms such as the exemplary predictive model described in the article by Jimenez, Rosa, et al. entitled “Difference between observed and predicted length of stay as an indicator of inpatient care inefficiency” International Journal for Quality in Health Care 1999; Volume 11, No. 5, pp. 375-384, the entirety of which is hereby incorporated by reference. Once the length of stay is predicted using actual data elements in the clinical records, this length of stay may be used in the predicted groups calculation. In other embodiments, additional data elements such as severity scores that are not actually used in the calculation of the group but may refine the prediction of the group may be employed to refine the predicted group.

At step 308, the one or more predicted billing groups for the patient calculated at step 306, may be displayed to a user. For example, a healthcare provider, such as a nurse or doctor, may be able to view the predicted billing code for the patient. This way, a healthcare provider can see the possible financial reimbursement for treatment for the patient during the time care is being provided and not only at the time of discharge. If a healthcare provider determines that the predicted billing group for the patient is not appropriate based on the treatment being provided to the patient, the predicted billing group can be modified by the entry of appropriate data elements and recalculation of the predicted billing group.

Thus, a change may be made in the predicted billing group before the patient is discharged so that the healthcare entity receives the appropriate financial reimbursement for the care provided to the patient. A predicted billing group may be recalculated during patient treatment much more easily than recalculating the final billing group for the patient after the patient has been discharged. In most instances, final billing groups are never recalculated and healthcare facility will not receive the appropriate financial reimbursement for the patient's stay and treatment.

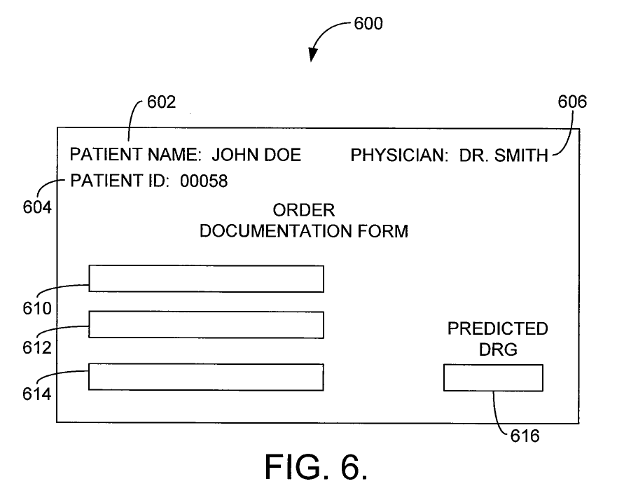

Alternatively, if the clinical treatment of a patient needs to be modified based on the predicted billing group, appropriate steps may be taken by the healthcare provider and/or facility to assure that the patient is receiving the appropriate care for his or her predicted billing group. With reference to FIG. 6, an exemplary screen is provided for displaying a predicted billing group 616 for patient 602. Along with the predicted billing group, data describing the billing group and amount of reimbursement for the group may also be displayed.

Referring again to FIG. 3, at step 309, in some instances the predicted billing group calculated for the patient may be utilized for the development of a patient care plan including procedures and tests that should be performed for the patient based on the predicted billing group. At step 310, the one or more predicted billing groups calculated for the patient and related data are stored in a computerized database such as the predicted financial records of the database 200 shown in FIG. 2. The billing group may be stored as a code, such as a DRG or APC code, or some other data form that represents a billing group. The related data may include the data elements, such as diagnosis and procedure codes used to calculated the predicted billing group, the user who performed the billing group calculation, the date the billing group was calculated, a priority ranking of all billing groups for the patient, an estimated reimbursement for the billing group, and the length of stay used to calculate the predicted billing group.

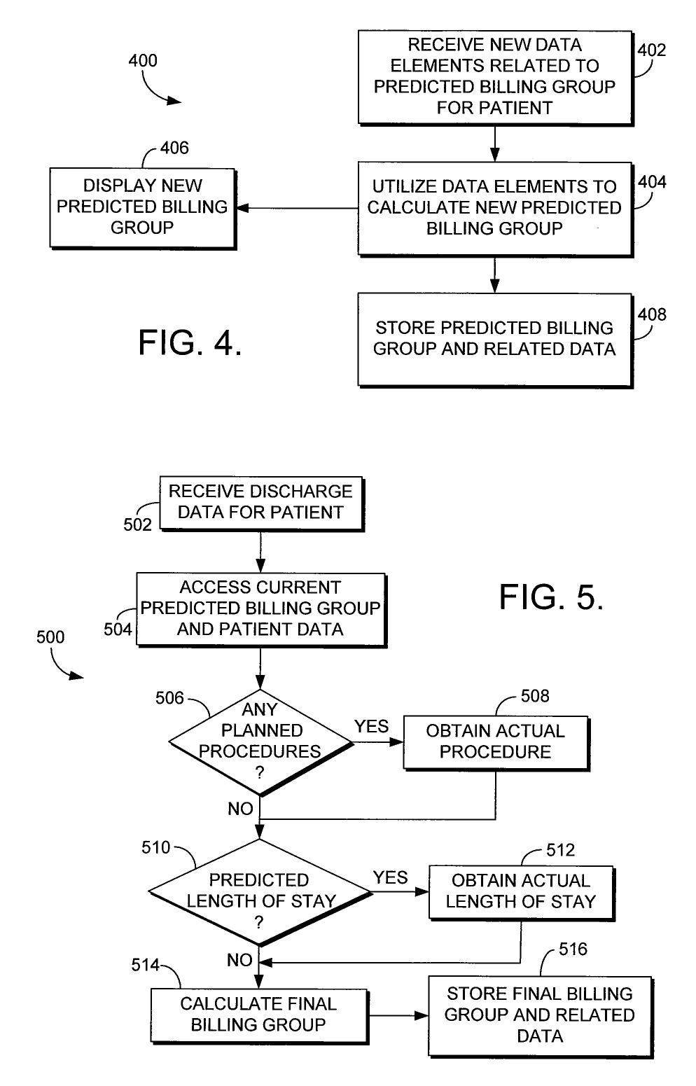

With reference to FIG. 4, a method for receiving new data elements for a patient and calculating one or more revised predicted billing groups for the patient is shown. At step 402, new data elements related to the predicted billing group for the patients are received. Additional data elements may include the estimated length of stay, primary and secondary diagnosis, information related to planned and performed procedures surgeries and tests, and the age and gender of the patient. For example, after admission and during treatment of the patient, if the primary diagnosis for the patient changes and a battery of new tests, these additional data elements are received by the system and are utilized to calculate a revised predicted billing group for the patient.

In one embodiment, if additional new data elements are entered for a patient the user may be prompted that the predicted billing group for the patient is no longer valid. In this embodiment, the user may request that a revised predicted billing group for the patient be calculated. In another embodiment, a revised predicted billing group for the patient is automatically recalculated.

At step 404, a revised predicted billing group utilizing the newly received data elements for the patient is calculated. At step 406, the revised predicted billing group is displayed to a healthcare provider. For example, healthcare provider, such as a nurse or doctor, views the revised predicted billing code for the patient as discussed above. At step 408, the revised predicted billing group for the patient and related data are stored in a computerized database such as the predicted financial records component of database 200 shown in FIG. 2. The related data may include the data elements, such as diagnosis and procedure codes, used to calculated the predicted billing group, an identifier of the user who performed the billing group calculation, the date the billing group was calculated, a priority ranking of all billing groups for the patient, an estimated reimbursement for the billing group, and the length of stay used to calculate the revised predicted billing group. The revised predicted billing group for the patient and related data are stored along with the previously calculated billing group for the patient and data related to the group so that historic information relating to the calculation of the predicted billing group may be accessed later.

With reference to FIG. 5, a method 500 for calculating a final billing group is shown. At step 502, discharge data for the patient is received. For instance, when a patient is to be discharged from a healthcare facility, this information is entered into the system. At step 504, a predicted billing group calculated for the patient is accessed along with patient data. For example, the most recently calculated predicted billing group is accessed along with related data for the predicted billing group. At step 506, it is determined whether any planned procedures were utilized to calculate the predicted billing group accessed. Procedures may include any tests, surgical consults or healthcare items performed for the patient. If so, at step 508, the actual procedures performed for the patient during the patient's care at the health facility are obtained. These actual procedure codes will be utilized to calculate the final billing group for the patient rather than the planned procedures. For example, if a CAT scan is ordered for a patient and utilized to calculate the predicted billing group, but a PET scan is actually performed, the PET scan is used to determine the billing group.

If, at step 506, it is determined that no planned procedures were utilized to calculate the predicted billing group for the patient, at step 510 it is determined whether the length of stay for the patient was predicted and utilized to calculate the predicted billing group. For example, at admission a data element was received that the patient's predicted length of stay was three nights but the patient actually stayed for five nights. Data such as the actual length of stay and performed procedures may be obtained from the patient's clinical record, such as the patient's electronic medical record.

If, at step 510, it is determined that the predicted length of stay was utilized to calculate the predicted group billing for the patient, at step 512, the actual length of stay for the patient in the healthcare facility is obtained. At step 514, the final billing group is calculated using the predicted billing group obtained for the patient along with any actual procedure data and actual length of stay, obtained for the patient. If planned procedures or predicted length of stay were not utilized to calculate the predicted billing group, then the predicted billing group becomes the final billing group. However, if any planned procedures or predicted length of stay were utilized to calculate the predicted billing group, then a new final billing group is calculated utilizing the predicted billing group and one or more of the actual procedure data for the patient and/or the actual length of stay for the patient. At step 516, the final billing group for the patient and related data is stored. For example, the final billing group for the patient and related data may be stored in the financial records 204 in database 200 of FIG. 2. Once the final billing group has been determined, a complete history of the billing groups and the data used to calculate the groups is available. This history demonstrates how the reimbursement varied through the stay and the data elements for the patient that affected the calculation of the billing group. This history is a valuable tool for care providers to analyze to understand the relationship between the care provided and documented and its impact on the group and level of reimbursement. The final billing group calculated may then be sent to Medicare or insurance companies for reimbursement.

With reference to FIG. 6, a screen 600 is shown for displaying an order documentation form for a patient 602. The order documentation form includes information for the patient, such as the patient name 602, the patient identification 604, and treating physician 606. The order documentation form also includes fields 610, 612, and 614 where information may be entered for the patient. The predicted billing group, such as the predicted DRG for a patient, may be displayed in field 616. Information related to the predicted billing group, such as a description of the billing group and amount of financial reimbursement for the group may also be displayed.

The present invention has been described in relation to particular embodiments, which are intended in all respects to illustrate rather than restrict. Alternative embodiments will become apparent to those skilled in the art that do not depart from its scope. Many alternative embodiments exist, but are not included because of the nature of this invention. A skilled programmer may develop alternative means for implementing the aforementioned improvements without departing from the scope of the present invention.

It will be understood that certain features and sub-combinations of utility may be employed without reference to features and sub-combinations and are contemplated within the scope of the claims. Furthermore, the steps performed need not be performed in the order described.

The invention claimed is:

1. A method in a computerized healthcare environment for calculating one or more predicted billing groups for a patient, wherein the method comprises:
receiving one or more data elements for a patient prior to the patient being discharged from a healthcare facility;
utilizing the one or more data elements to calculate one or more predicted billing groups for the patient; and
storing the one or more calculated predicted billing groups for the patient.

2. The method of claim 1, wherein one or more data elements are selected from the group consisting of estimated length of stay for the patient, primary diagnosis of the patient, secondary diagnosis of the patient, planned procedures, performed procedures, age of patient and sex of patient and combinations thereof.

3. The method of claim 2, wherein the length of stay is estimated using clinical patient data and an algorithm.

4. The method of claim 3, wherein the one or more data elements are coded.

5. The method of claim 4, wherein the one or more billing groups are diagnostic related group codes.

6. The method of claim 1, wherein the one or more billing groups are ambulatory payment classification codes.

7. The method of claim 1, further comprising:
receiving one or more additional data elements for a patient prior to the patient being discharged from a healthcare facility; and
utilizing the one or more additional data elements and the one or more predicted billing groups for the patient to calculate one or more revised predicted billing groups for the patient.

8. The method of claim 1, further comprising:
displaying the one or more predicted billing groups for the patient to a healthcare provider.

9. The method of claim 1, further comprising:
storing information related to the one or more predicted billing groups calculated for the patient.

10. The method of claim 9, wherein the information related to the one or more predicted billing groups is selected from the group consisting of diagnosis codes and procedure codes used to calculated the predicted billing group, an identifier of the user who performed the billing group calculation, date the billing group was calculated, priority ranking of all billing groups for the patient, estimated reimbursement for the billing group, and length of stay used to calculate the predicted billing group and combinations thereof.

11. The method of claim 1, wherein the method is stored on one or more computer readable media.

12. A method in a computerized healthcare environment for calculating one or more final billing groups for a patient, the method comprising:
accessing one or more predicted billing groups for a patient; and
utilizing the one or more predicted billing groups for the patient for calculating one or more final billing groups for the patient.

13. The method of claim 12, further comprising:
determining whether the one or more predicted billing groups were calculated using estimated length of stay data for the patient.

14. The method of claim 13, wherein if the one or more predicted billing groups were calculated using estimated length of stay data for the patient, accessing actual length of stay data for the patient.

15. The method of claim 14, further comprising:
utilizing the actual length of stay data to calculate the one or more final billing groups for the patient.

16. The method of claim 12, further comprising:
determining whether the one or more predicted billing groups were calculated using planned procedure data for the patient.

17. The method of claim 16, wherein if the one or more predicted billing groups were calculated using planned procedure data for the patient, accessing the actual procedure data performed for the patient.

18. The method of claim 17, further comprising:
utilizing the actual procedure data to calculate the one or more final billing groups for the patient.

19. The method of claim 12, wherein the method is stored on one or more computer readable media.

20. A computer system healthcare environment for calculating one or more predicted billing groups for a patient, wherein the system comprises:
a receiving component for receiving one or more data elements for a patient prior to the patient being discharged from a healthcare facility;
a utilizing component for utilizing the one or more data elements to calculate one or more predicted billing groups for the patient;
a storing component for storing the one or more calculated predicted billing groups for the patient.

21. The system of claim 20, wherein one or more data elements are selected from the group consisting of estimated length of stay for the patient, primary diagnosis of the patient, secondary diagnosis of the patient, planned procedures, performed procedures, age of patient and sex of patient and combinations thereof.

22. The system of claim 20, wherein the one or more billing groups are diagnostic related group codes.

23. The system of claim 20, wherein the receiving component receives one or more additional data elements for a patient prior to the patient being discharged from a healthcare facility and the utilizing component utilizes the one or more additional data elements and the one or more predicted billing groups for the patient to calculate one or more revised predicted billing groups for the patient.

24. The system of claim 20, further comprising:
a displaying component for displaying the one or more predicted billing groups for the patient to a healthcare provider.

25. A computer system in a healthcare environment for calculating one or more final billing groups for a patient, the system comprising:
an accessing component for accessing one or more predicted billing groups for a patient; and
a utilizing component for utilizing the one or more predicted billing groups for the patient for calculating one or more final billing groups for the patient.

26. The method of claim 25, further comprising:
a determining component for determining whether the one or more predicted billing groups were calculated using estimated length of stay data for the patient.

27. The system of claim 25, wherein if the determining component determines the one or more predicted billing groups were calculated using estimated length of stay data for the patient, the accessing component accesses actual length of stay data for the patient.

28. The system of claim 27, wherein the utilizing component utilities the actual length of stay data to calculate the one or more final billing groups for the patient.

29. The system of claim 25, further comprising:
a determining component for determining whether the one or more predicted billing groups were calculated using planned procedure data for the patient.

30. The system of claim 29, wherein if the determining component determines the one or more predicted billing groups were calculated using planned procedure data for the patient, the accessing component accesses the actual procedure data performed for the patient.

31. The system of claim 30, wherein the utilizing component utilizes the actual procedure data to calculate the one or more final billing groups for the patient.
